(19) United States
(12) Patent Application Publication
(10) Pub. No.: US 2021/0186638 A1
(43) Pub. Date: Jun. 24, 2021

(54) SURGICAL ROBOT SYSTEM AND SURGICAL INSTRUMENT THEREOF

(71) Applicant: MICROPORT (SHANGHAI) MEDBOT CO., LTD., Shanhai (CN)

(72) Inventors: Yuhui XIA, Shanghai (CN); Chao HE, Shanghai (CN); Tao LI, Shanghai (CN); Jiayin WANG, Shanghai (CN); Yunlei SHI, Shanghai (CN)

(21) Appl. No.: 16/981,514

(22) PCT Filed: Mar. 5, 2019
(86) PCT No.: PCT/CN2019/077059

§ 371 Date: Sep. 16, 2020

(30) Foreign Application Priority Data
Mar. 16, 2018 (CN) 201810218810.2

(51) Int. Cl. A61B 34/00; A61B 34/37; A61B 34/35; A61B 90/00

(52) U.S. Cl. CPC A61B 34/76; A61B 34/37; A61B 2034/301; A61B 90/06; A61B 34/35

(57) ABSTRACT

The present invention provides a surgical robot system and a surgical instrument thereof, which can measure a contact force acting on a terminal end of the surgical instrument with enhance precision and accuracy. The surgical instrument includes a mechanical structure, a sleeve and a force sensor. The mechanical structure includes a shaft and an end effector connected to a terminal end of the shaft. The sleeve fixedly or detachably sleeves over the terminal end of the shaft. The force sensor includes a sensitive element and a peripheral measurement module connected thereto. The sensitive element is arranged on the sleeve and configured to acquire strain information of the sleeve. The peripheral measurement module is configured to obtain stress measured by the sensitive element based on the strain information acquired thereby, and to obtain both an equivalent resultant force and an equivalent resultant moment of an action force exerted by the shaft on the sleeve based on the stress measured by the sensitive element, the position of the sensitive element and the direction of the sensitive axis of the sensitive element, thereby obtaining a contact force acting on the end effector of the surgical instrument.

### TECHNICAL FIELD

The present invention relates to the technical field of medical devices and, more specifically, to a surgical robot system and a surgical instrument thereof.

### BACKGROUND

A robotically-assisted surgical system usually adopts a master-slave control structure consisting of a master (surgeon side) and a slave (patient side). The master is usually capable of multi-dimensional movement based on components such as robotic arms, multi-dimensional mice, joysticks, etc., while the slave is usually a robotic arm with multiple degrees of freedom holding, at an end thereof, surgical instruments (e.g., needle holders, electrocoagulation forceps, electrosurgical cautery probes, tissue graspers, scissors, etc.) During the operation, based on a 3D image of a target surgical site from an endoscope displayed on a display device, the surgeon operates the master so that the slave follows motion of the master to drive the surgical instruments to perform various surgical operations, e.g., retracting tissue, suturing, grasping a blood vessel, dissecting, cauterizing, coagulating tissue, etc.

Such a robotically-assisted surgical system offers the following advantages:

First, it is designed to perform a minimally invasive intervention through a small surgical incision made in a patient's body, which results in less trauma to the patient and hence reduced postoperative pain, rapid recovery, a lower risk of infection and a minimized need for blood transfusion.

Second, it operates much more precisely and stably by the system when compared to human hands. For example, the system can eliminate physiological tremor, and even tiny capillaries can be clearly viewed in the 3D visual operative field, allowing high surgical safety.

However, since robotically-assisted system is based on tele-operation mode which fails in enabling the surgeon to have a direct tactile sensation, identifying tissue toughness, feeling vascular pulsation, as well as lack of feedback to the gripping and pressing actions. A feedback may occur only when a serious collision to the human body by the surgical instrument occurs, and the sensitive feedbacks are difficult to occur in the case of slight touches. Therefore, imparting to such a robot the ability to adjust motion of the slave according to variation of a force exerted on the terminal end of a surgical instrument that is being brought into contact with a body tissue and to provide the surgeon with appropriate force feedback indications can significantly augment the system's safety.

Most traditional force feedback mechanisms rely on torque sensors disposed at joints of the robot, and a contact torque on the terminal end can be calculated based on the torque on the joints. This approach, however, suffers from a number of disadvantages in that the calculated contact force cannot well reflect in real time what is actually going on. Moreover, the detectability of a minor contact force requires augmenting the torque sensors' measurement accuracy and resolution, which will makes the sensor having a high resolution sensitive to noises, easy to cause signal distortions and result in expensive costs. Further, transmission in the surgical instrument is usually achieved by wires, which are obviously not suitable for arranging tension sensors thereon.

U.S. Pat. No. 8,491,574B2 discloses a solution for the above problems, in which a strain sensitive element capable of directly measuring an external force is embedded in the terminal end of a surgical instrument. This, however, is still associated with the following shortcomings:

First, it is inevitable that a single surgical operation needs a lot of surgical instruments. When each of these surgical instruments is provided with a respective sensor, it will be difficult to ensure measurement consistency across the different sensors. Even when each sensor is calibrated, accurate recording of all the instruments' physical parameters is difficult.

Second, physical properties of the sensitive elements are susceptible to factors such as temperature, humidity and atmospheric pressure, and the performances of the sensitive elements vary between environments such as, inside and outside the patient's body. Therefore, it is difficult for such sensitive elements to describe forces acting on the surgical instruments in a consistent way.

Third, the surgical instruments themselves are consumable and can be generally used for up to 10 times, but the sensors thereon are reusable for more times. Obviously, scraping the surgical instrument together with the sensors is not an economical practice.

Patent publication WO2009079301A1 discloses a force sensor apparatus including an inner tube and an outer tube disposed over the inner tube. Strain gauges are disposed on the inner tube, and a proximal end of the inner tube is connected to a surgical instrument so that the strain gauges on the inner tube can sense a force acting on the surgical instrument. However, this force sensor apparatus is associated with a number of problems such as poorer measurement consistency and lower measurement accuracy.

Patent publication CN104764552A discloses a force-sensitive sensor for sensing surgical forces, which is complicated in structure with multiple beams. In addition, since strain gauges are not directly arranged on components that undergo strains, the sensing accuracy thereof is poorer. Further, this force-sensitive sensor suffers from the following problems: due to the absence of a precise mathematical model, the measurement can rely only on calibration, making it impossible to obtain accurate results; and the axial force measurement is not available due to its structural design.

### SUMMARY OF THE INVENTION

It is an objective of the some embodiments of the present invention to provide a surgical robot system and a surgical instrument thereof, which can solve one or more of the above-described problems in the prior art, including low accuracy in measuring a contact force on the terminal end of a surgical instrument and unnecessary scrappage of sensors.

The above and related objectives are achieved by providing a surgical instrument, comprising a mechanical structure, a sleeve and a force sensor;

wherein the mechanical structure comprises a shaft and an end effector connected to a terminal end of the shaft;

the sleeve fixedly or detachably sleeves over the terminal end of the shaft;

the force sensor comprises at least one sensitive element and a peripheral measurement module connected to the sensitive element, the sensitive element is disposed on the sleeve and configured to obtain a strain information of the sleeve;

the peripheral measurement module is configured to obtain a stress measured by the sensitive element based on the strain information obtained by the sensitive element and obtain both an equivalent resultant force and an equivalent resultant moment of an action force exerted on the shaft to the sleeve based on the stress measured by the sensitive element, the position of the sensitive element and the direction of a sensitive axis of the sensitive element, thereby obtaining a contact force acting on the end effector of the surgical instrument.

Optionally, the sleeve comprises a hollowed-out feature extending in a circumferential direction, the hollowed-out feature comprising a plurality of compliant elements and openings each located between adjacent two of the compliant elements, the compliant elements being configured to connect an upper section of the sleeve above the hollowed-out feature to a lower section of the sleeve below the hollowed-out feature, and wherein the at least one sensitive element is arranged on the compliant elements and configured to measure a stress on the compliant elements.

Optionally, the compliant elements have a modulus of elasticity that is not higher than a modulus of elasticity of the shaft.

Optionally, each of the compliant elements comprises an inner surface, an outer surface and side surfaces, and wherein the at least one sensitive element is arranged on the inner surfaces, outer surfaces or side surfaces of the compliant elements.

Optionally, the hollowed-out feature is arranged around an axial middle of the sleeve.

Optionally, the sensitive axis of the sensitive element is arranged to be parallel or perpendicular to an axial direction of the sleeve.

Optionally, the sleeve fixedly sleeves over the terminal end of the shaft by bonding, an interference fit or insertion.

Optionally, the sleeve detachably sleeves over the terminal end of the shaft by thread connection, a latch connection or a snap fitting.

Optionally, the sleeve has one end flush with the terminal end of the shaft.

Optionally, the peripheral measurement module comprises, communicatively connected to one another in series, a data acquisition unit, a signal conditioning unit and a calculation and output unit; wherein the data acquisition unit is configured to acquire signals output from the sensitive element, the signal conditioning unit is configured to condition the signals output from the sensitive element, and the calculation and output unit is configured to perform a calculation based on the conditioned signals to obtain the contact force on the end effector.

Optionally, the stress measured by the sensitive element is given by:

fᵢ=g(εᵢ)=kᵢ·εᵢ+f(εᵢ) i=1,2, . . . ,n,

where i represents an i-th sensitive element; fᵢ, a stress measured by the i-th sensitive element; εᵢ, a strain of the compliant element, on which the i-th sensitive element is arranged, along the direction of the sensitive axis of the i-th sensitive element; g(⋅), a function describing a relationship between the stress and the strain; kᵢ, a stress-strain factor of the compliant element, on which the i-th sensitive element is arranged, along the direction of the sensitive axis of the i-th sensitive element; and f(εᵢ), a non-linear compensation term for the stress and the strain.

Optionally, the contact force comprises an equivalent resultant force F_{q} and an equivalent resultant moment M_{q};

the peripheral measurement module is configured to establish a coordinate system {p} for the sleeve thereon, a coordinate system {q} for the surgical instrument at the terminal end thereof, and descriptors of the position and posture of the coordinate system {p} in the coordinate system {q};

the peripheral measurement module is further configured to obtain a descriptor of the stress measured by the sensitive element in the coordinate system {q} based on the descriptor of the posture of the coordinate system {p} in the coordinate system {q}, a descriptor of the direction of the sensitive axis of the sensitive element in the coordinate system {p} and the stress measured by the sensitive element, thereby obtaining the equivalent resultant force F_{q};

the peripheral measurement module is further configured to obtain a descriptor of the position of the sensitive element in the coordinate system {q} based on the descriptors of the position and posture of the coordinate system {p} in the coordinate system {q} and a descriptor of the position of the sensitive element in the coordinate system {p}; and

the peripheral measurement module is further configured to obtain a descriptor of a moment of force acting on the sleeve in the coordinate system {q} based on the descriptor of the stress measured by the sensitive element in the coordinate system {q}, thereby obtaining the equivalent resultant moment M_{q}.

Optionally, a descriptor of the stress measured by the sensitive element in the coordinate system {p} is given by:

(fₓᵢ,f_{yi},f_{zi})^{T}=fᵢeᵢ=fᵢ·(eₓᵢ,e_{yi},e_{zi})^{T} i=1,2, . . . ,n,

where eᵢ is a unit vector denoting a descriptor of the direction of the sensitive axis of the i-th sensitive element in the coordinate system {p}; fᵢ, the stress measured by the i-th sensitive element; fₓᵢ, f_{yi}, f_{zi}, components of the fᵢ along axes of the coordinate system {p}; and eₓᵢ, e_{yi}, e_{zi}, components of eᵢ along axes of the coordinate system {p}.

Optionally, the descriptor of the stress measured by the sensitive element in the coordinate system {q} is given by:

fᵢ=^{q}R(fᵢeᵢ)

where fᵢ represents a descriptor of the stress measured by the i-th sensitive element in the coordinate system {q}, and ^{q}R represents the descriptor of the posture of the coordinate system {p} in the coordinate system {q}, and wherein

the equivalent resultant force F_{q} is given by:

$${F}_{q} = diag ( 1 \text{/} {n}_{x} , 1 \text{/} {n}_{y} , 1 \text{/} {n}_{z} ) {\sum }_{i = 1}^{n} {f}_{i} ,$$

where diag(•) represents a diagonal matrix with elements in the vector • as diagonal elements, and nₓ,n_{y},n_{z} represent numbers of redundant forces in the directions of the x-axis, y-axis and z-axis, respectively.

Optionally, the descriptor of the position of the sensitive element in the coordinate system {q} is given by:

rᵢ=rₚ+^{q}R^{p}rᵢ

where rᵢ is a descriptor of the position of the i-th sensitive element in the coordinate system {q}; ^{p}rᵢ, a descriptor of the position of the i-th sensitive element in the coordinate system {p}; ^{q}R, the descriptor of the posture of the coordinate system {p} in the coordinate system {q}; and rₚ, a descriptor of the position of the coordinate system {p} in the coordinate system {q}.

Optionally, the equivalent resultant moment M_{q} is given by:

$${M}_{q} = diag ( 1 \text{/} {n}_{x}^{′} , 1 \text{/} {n}_{y}^{′} , 1 \text{/} {n}_{z}^{′} ) {\sum }_{i = 1}^{n} {r}_{i} \times {f}_{i}$$

where nₓ′,n_{y}′,n_{z}′ represent the numbers of redundant moments in the directions of the x-axis, y-axis and z-axis, respectively.

Optionally, the hollowed-out feature comprises four compliant elements that are arranged along the circumferential direction; the force sensor comprises four sensitive elements, each arranged on an outer surface of a respective one of the compliant elements; the descriptors e₁, e₂, e₃, e₄ of the directions of the sensitive axes of the four sensitive elements in the coordinate system {p} are all (1,0,0)^{T};

the descriptors of the stress measured by the four sensitive elements in the coordinate system {q} are given as:

$$\{ \begin{matrix}{f}_{1} = {( {f}_{x 1} 0 \; 0 )}^{T} = {f}_{1} {e}_{1} \\ {f}_{2} = {( {f}_{x 2} 0 \; 0 )}^{T} = {f}_{2} {e}_{2} \\ {f}_{3} = {( {f}_{x 3} 0 \; 0 )}^{T} = {f}_{3} {e}_{3} \\ {f}_{4} = {( {f}_{x 4} 0 \; 0 )}^{T} = {f}_{4} {e}_{4}\end{matrix}$$

where fₓ₁, fₓ₂, fₓ₃, fₓ₄ respectively represent stress measured by the first to fourth sensitive elements in the direction of an x-axis of the coordinate system {q}; and

the equivalent resultant force F_{q} is given by:

F_{q}=(f₁+f₂+f₃+f₄).

18. The surgical instrument of claim 17, wherein:

^{q}R, the descriptor of the posture of the coordinate system {p} in the coordinate system {q}, is an identity matrix; and

the positions of the sensitive elements are measured as respective center positions, which are described in the coordinate system {q} as:

$$\{ \begin{matrix}{r}_{1} = {( {x}_{1} {y}_{1} {z}_{1} )}^{T} = {r}_{p} + {}_{}^{p}{r}{}_{1}^{} \\ {r}_{2} = {( {x}_{2} {y}_{2} {z}_{2} )}^{T} = {r}_{p} + {}_{}^{p}{r}{}_{2}^{} \\ {r}_{3} = {( {x}_{3} {y}_{3} {z}_{3} )}^{T} = {r}_{p} + {}_{}^{p}{r}{}_{3}^{} \\ {r}_{4} = {( {x}_{4} {y}_{4} {z}_{4} )}^{T} = {r}_{p} + {}_{}^{p}{r}{}_{4}^{}\end{matrix}$$

where ^{p}r₁, ^{p}r₂, ^{p}r₃, ^{p}r₄ are descriptors of the center positions of the four sensitive elements in the coordinate system {p}; rₚ, the descriptor of the position of the coordinate system {p} in the coordinate system {q}; x₁, x₂, x₃, x₄, x-axis coordinates of the center positions of the first to fourth sensitive elements in the coordinate system {q}; y₁, y₂, y₃, y₄, y-axis coordinates of the center positions of the first to fourth sensitive elements in the coordinate system {q}; z₁, z₂, z₃, z₄, z-axis coordinates of the center positions of the first to fourth sensitive elements in the coordinate system {q}; and r₁, r₂, r₃, r₄, descriptors of the positions of the first to fourth sensitive elements in the coordinate system {q}.

Optionally, the equivalent resultant moment M_{q} is given as:

M_{q}=(r₁×f₁+r₂×f₂+r₃×f₃+r₄×f₄).

Further, the present invention also provides a surgical robot system, comprising a slave, the slave comprising: a robotic arm; and the surgical instrument as defined above, the robotic arm comprising a terminal end detachably connected to the surgical instrument, the robotic arm being configured to drive the surgical instrument to move about a remote center of motion.

Optionally, the surgical robot system further comprises a master and a control unit, the master comprising a force indicator;

wherein the control unit is communicatively connected to both the master and the slave; the control unit is configured to obtain an information about contact force acting on the end effector from the force sensor of the surgical instrument and transmit the information to the force indicator, the force indicator is configured to show the information about the contact force acting on the end effector.

Optionally, the slave further comprises:

an endoscope; and

an endoscope holder detachably connected to the endoscope;

wherein the peripheral measurement module of the surgical instrument is configured to establish a coordinate system {p} for the sleeve, a coordinate system {q} for the surgical instrument and a coordinate system {e} for the endoscope, and to obtain, from a matrix ^{e}R describing a rotation from the coordinate system {q} to the coordinate system {e} and a position vector r=(rₓ r_{y} r_{z})^{T} from the coordinate system {q} to the coordinate system {e}, a descriptor of the contact force acting on the end effector in the coordinate system {e} as:

$$( \begin{matrix}{}^{e} F \\ {}^{e} M\end{matrix} ) = ( \begin{matrix}{}^{e} R & 0 \\ - {S}^{T} ( {}^{e} r ) {}^{e} R & {}^{e} R\end{matrix} ) ( \begin{matrix}{F}_{q} \\ {M}_{q}\end{matrix} )$$

where ^{e}F denotes a resultant force in the coordinate system {e}; ^{e}M, a resultant moment in the coordinate system {e}; S(^{e}r), an anti-symmetric matrix corresponding to the vector ^{e}r, F_{q}, the equivalent resultant force of the contact force acting on the end effector; M_{q}, the equivalent resultant moment of the contact force acting on the end effector; and

$$S ( {}^{e} r ) = ( \begin{matrix}0 & - {r}_{z} & {r}_{y} \\ {r}_{z} & 0 & - {r}_{x} \\ - {r}_{y} & {r}_{x} & 0\end{matrix} ) .$$

In summary, the surgical robot system and the surgical instrument thereof provided in the present invention provide at least one of the following advantages:

First, no clearance occurs between the sleeve and the mechanical structure of the surgical instrument. This is conducive to improved measurement accuracy.

Second, for a surgical operation requiring the use of several surgical instruments, a specific calibration process can be performed on each of the surgical instruments, avoiding the influence of instrument material inconsistency on the measurement.

Third, identical sleeve can be used on different surgical instruments which transmits deformations of these surgical instruments. Compared to sensitive elements directly attaching to the terminal ends of the surgical instruments, the use of the sensitive elements with the sleeve can avoid measurement errors that may arise from measurement inconsistency between the different sensitive elements on the different surgical instruments, helping in achieving a higher degree of measurement precision and accuracy.

Fourth, coupling the sensitive elements to the surgical instruments with the sleeves can avoid scrappage of the sensitive elements together with the surgical instruments. The surgical instruments are consumable, while the sensitive elements are reusable. Obviously, scraping the sensitive elements together with the surgical instruments is not an economic practice and will lead to increased usage cost.

In a preferred embodiment of the present invention, a plurality of sensitive elements are arranged on the compliant elements of the sleeve in a predetermined manner, and each of the sensitive elements has predetermined coordinates in the coordinate system for the surgical instrument. Based on the predetermined coordinates, the positions of the sensitive elements on the compliant elements can be determined, thus allowing one or more dimensional measurement of a contact force acting on the terminal end of the surgical instrument. This entails an easier and simpler contact force measurement approach, in which one or more dimensional contact force measurement is achievable only by adjusting the positions of the sensitive element relative to the coordinate system for the surgical instrument.

### BRIEF DESCRIPTION OF THE DRAWINGS

FIG. 1 shows a schematic of a surgical robot system according to an embodiment of the present invention.

FIG. 2 is a schematic diagram illustrating the mechanical structure of a surgical instrument according to an embodiment of the present invention.

FIG. 3 schematically illustrates the surgical instrument that is performing a surgical operation according to an embodiment of the present invention.

FIG. 4a is a schematic diagram of a force sensor and a sleeve in the surgical instrument according to an embodiment of the present invention.

FIG. 4b is a structural block diagram of a peripheral measurement module in the surgical instrument according to an embodiment of the present invention.

FIG. 5a is a schematic diagram of a sleeve disposed over a shaft in the surgical instrument according to an embodiment of the present invention.

FIG. 5b is a schematic enlarged partial view of the sleeve and the shaft of FIG. 5a.

FIG. 6a is a schematic diagram of the sleeve that has not been assembled with the shaft according to an embodiment of the present invention.

FIG. 6b is a schematic diagram of the sleeve that has not been assembled with the shaft according to another embodiment of the present invention.

FIG. 7a is a schematic diagram of the surgical instrument that is being stressed on one side and deformed according to an embodiment of the present invention.

FIG. 7b is a schematic diagram illustrating analysis on a force acting on the terminal end of the surgical instrument and measurement principle thereof in accordance with an embodiment of the present invention.

FIG. 8 is a schematic diagram illustrating force measurement principles of a three-dimensional force sensor according to an embodiment of the present invention.

In these figures,

1 denotes a surgical cart; 2, a robotic arm; 3, a surgical instrument; 301, a power module; 302, a shaft; 303, an end effector; 304, a roll joint; 305, a pitch joint; 306, 306′, a yaw joint; 4, an endoscope; 5, an imaging system; 6, a surgeon; 7, a master manipulator; 8, a patient;

400, a force sensor; 401, a sleeve; 4011, a compliant element; 402, a sensitive element; 403, a peripheral measurement module; 4031, a data acquisition unit; 4032, a signal conditioning unit; and 4033, calculation and output unit.

### DETAILED DESCRIPTION

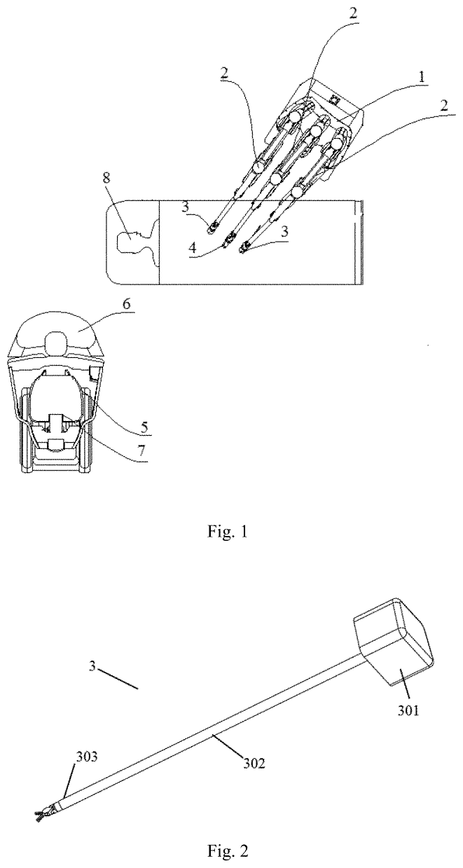

Objectives, advantages and features of the present invention will become more apparent upon reading the following description of the surgical robot system and the surgical instrument thereof proposed in the invention in conjunction with the accompanying drawings, i.e., FIGS. 1 through 8. Note that the figures are provided in a very simplified form not necessarily presented to scale, with their only intention to facilitate convenience and clarity in explaining a few embodiments disclosed herein. As used herein, a “terminal end” or “distal end” of a product refers to the end thereof farther away from a user operating the product, while a “proximal end” refers to the end of the product closer to the user.

FIG. 1 is a schematic of a surgical robot system according to an embodiment of the present invention. As shown in FIG. 1, the surgical robot system includes a slave including a surgical cart 1, robotic arms 2, a surgical instrument 3 and an endoscope 4. The surgical cart 1 serves as a base of the slave and supports all mechanical components thereof. Preferably, the surgical cart 1 is able to move on the ground so as to bring the slave closer to or away from a patient 8.

The robotic arm 2 is mounted on the surgical cart 1 and has multiple degrees of freedom, which make it able to move within a certain spatial range. When the surgical cart 1 moves into the vicinity of the patient 8, the robotic arm 2 can be adjusted to place the surgical instrument 3 at a planned operating position. The surgical instrument 3 is detachably mounted at a terminal end of one of the robotic arms 2 and is configured as an actuating mechanism of the slave, which is able to pivot about a remote center of motion (RCM) under the drive of the robotic arm 2. The surgical instrument 3 has an end effector 303 configured to be inserted into the patient's body to treat a lesion therein.

The endoscope 4 is mounted at a terminal end of another robotic arm 2 (which is therefore also referred to herein as an “endoscope holder”) and is configured to collect image information in the surgical environment, including but not limited to information about the lesion and information about the position and posture of the surgical instrument 3. Additionally, the endoscope 4 mounted on the robotic arm 2 is communicatively connected to the master as detailed below configured to display in real time the collected image information in the surgical environment. The endoscope 4 may be stereoscopic or not, without limitation.

FIG. 2 is a schematic diagram illustrating the mechanical structure of a surgical instrument according to an embodiment of the present invention. As shown in FIG. 2, the mechanical structure of the surgical instrument 3 includes a power module 301, a shaft 302, a transmission mechanism and an end effector 303. The transmission mechanism may be a wire transmission mechanism that is housed in the shaft 302 and coupled to both the power module 301 and the end effector 303. The power module 301 may be disposed at a proximal end of the shaft 302, and the end effector 303 may be disposed at a distal end of the shaft 302.

The power module 301 is configured to provide the end effector 303 with a driving force, which is transmitted to the end effector 303 through the transmission mechanism and configured to cause the end effector 303 to perform a multi-dimensional movement such as pivoting, opening/closing, etc. Specific opreations that the end effector 303 can perform on the lesion in the patient may include cutting, probing, grasping, etc. Accordingly, the end effector 303 may be implemented as a scissor, a forcep, a probe, etc. According to the present invention, the power module 301 may be self-powered. In this case, for example, the power module 301 may include a power source and a driver. Alternatively, the power module 301 may also be externally powered so as to provide the end effector 303 with a driving force. In this case, for example, the power module 301 may include an interface connected to an external power supply and, preferably, may further include a power distribution module.

As shown in FIG. 1, the surgical robot system further includes a master including an imaging system 5 and a master manipulator 7. During a surgical operation, while monitoring the movement of the surgical instrument 3 that is indicated by image information collected by the endoscope 4 and displayed on the imaging system 5, the surgeon 6 can control the surgical instrument 3 to perform multi-dimensional movements as necessary for the operation, such as pitching, yawing, rotating, opening/closing, etc., by manipulating the master manipulator 7.

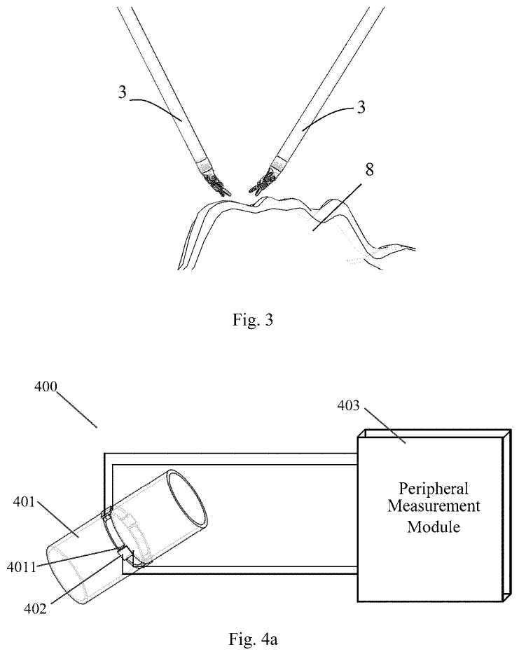

FIG. 3 schematically illustrates a surgical instrument performing surgical operations according to an embodiment of the present invention that is performing a surgical operation. As shown in FIG. 3, the surgeon 6 can manipulate one or more such surgical instruments 3 (identical or not) through the master manipulator 7 to perform cutting, probing, grasping and other actions as required by a surgical operation.

Obviously, the control of master manipulator 7 over the surgical instrument(s) 3 is the basis for master-slave control of the surgical robot system. However, since such control is based on tele-operation, the surgeon cannot directly feel the magnitude of an applied force, which is not favorable for the surgeon to take an appropriate action based on the strength of a contact force. To overcome this, the present invention provides a surgical instrument with force feedback capabilities, which allow the surgeon 6 to feel a force acting on a terminal end of the surgical instrument.

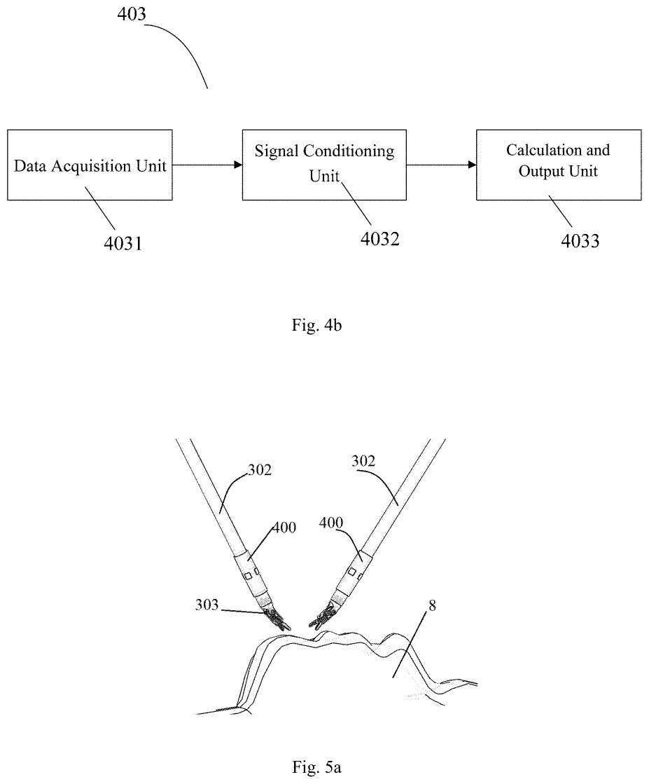

FIG. 4a is a schematic diagram of a force sensor and a sleeve in the surgical instrument according to an embodiment of the present invention. As shown in FIG. 4a, in addition to the above-discussed mechanical components, the surgical instrument 3 further includes the sleeve 401 and the force sensor 400. The force sensor 400 includes sensitive elements 402 and a peripheral measurement module 403. The sleeve 401 includes a hollow lumen with an inner diameter that matches an outer diameter of the shaft 302 in the surgical instrument 3. Further, the sleeve 401 includes a hollowed-out feature extending circumferentially which forms a closed circumferential ring across the sleeve 401. Here, the “circumferential extension” of the hollowed-out feature across the sleeve 401 means that a middle cross-section of the hollowed-out feature may be either perpendicular to or inclined with respect to an axis of the sleeve 401. The hollowed-out feature includes a plurality of compliant elements 4011 spaced from one another by openings each between adjacent two of the compliant elements 4011. As such, the compliant elements 4011 couple an upper section of the sleeve (above the hollowed-out feature) to a lower section thereof (below the hollowed-out feature). With this design, an external force is transmitted from one end of the sleeve to the other end via the compliant elements 4011, the magnitude and direction of the external force can be determined by measuring a force transmitted by the respective compliant elements 4011. This design can facilitate external force measurement with reduced algorithm complexity and increased accuracy.

In the preferred embodiment shown in FIG. 8, the hollowed-out feature that extends circumferentially on the sleeve 401 includes four compliant elements 4011 distributed in a centrally symmetric manner and form a cross shape. The present invention is not limited to any particular method for fabricating the hollowed-out feature and the compliant elements 4011. In an exemplary embodiment, the sleeve 401 with the hollowed-out feature is fabricated by removing undesired portions from a tubular blank mechanically, e.g., by wire cutting or electric sparking. In an alternative embodiment, the sleeve may be fabricated by fixing the compliant elements 4011 to the upper and lower sections. Therefore, the compliant elements 4011 may be made either of the same material as, or a different material from, the remainder of the sleeve. In order for improved force measurement sensitivity to be achieved, which can facilitate the sensitive elements 402 to sense an external force acting on the surgical instrument, the compliant elements 4011 may be made of a material selected to have a modulus of elasticity not higher than that of the shaft 302, such as rubber.

Each compliant element 4011 includes an inner surface, an outer surface and side surfaces. The sensitive elements 402 may be disposed to the inner, outer or side surfaces of the compliant elements 4011 in the sleeve 401. Preferably, each of the sensitive elements 402 has a sensitive axis extending in the axial or circumferential direction of the sleeve 401 and can sense the magnitudes of forces acting on the compliant elements 4011 along the sensitive axis. Here, the extension direction of the “sensitive axis” refers to the only direction in which the sensitive elements 402 are sensitive to strain. That is, the sensitive elements 402 are insensitive in any other direction. In some preferred embodiments of the present invention, the sensitive axis of the sensitive element 402 is set to be parallel to the axial direction of the sleeve 401. In practice, the sensitive elements 402 may be so mounted that their sensitive axis extends parallel to the axial direction of the sleeve 401, allowing them to sense strains of the compliant elements 4011 in this direction.

The sensitive elements 402 may be selected as strain gauges, fiber optics or other components, according to their application conditions and measurement requirements. The number of the sensitive elements 402 may be selected, and their arrangement may be designed, in a flexible manner depending on the type of contact force to be measured and the reliability of the sensor. The sensitive elements 402 may be attached on the inner, outer or side surfaces of the compliant elements 4011 in the sleeve 401 in a flexible manner depending on how the attachment is accomplished and on whether it is difficult. Preferably, the hollowed-out feature and hence the compliant elements 4011 are arranged in a portion of the sleeve 401 tending to experience a greater strain (such as an axial middle of the sleeve 401). Preferably, the plurality of sensitive elements 402 arranged on the compliant elements 4011 are capable of one- or multi-dimensional contact force measurement, e.g., three- or six-dimensional contact force measurement. More preferably, each of the compliant elements 4011 is provided (e.g., on the outer surface thereof) with one sensitive element 402.

It is to be noted that structural strength of the sleeve 401 should be taken into account in its practical design. For example, although it is theoretically feasible for the hollowed-out feature to include only one compliant element 4011, structural strength of the sleeve 401 in this case may be too weak to allow it to transmit an external force in a desirable way or to prevent significant plastic deformations of the compliant element 4011, which are detrimental to measurement accuracy. Therefore, the embodiment of FIG. 8 with four compliant elements 4011 that are arranged equiangularly on the circumference of the hollowed-out feature and thus at the four ends of a symmetric cross is preferred.

The peripheral measurement module 403 is communicatively (e.g., electrically) connected to the sensitive element 402 and configured to collect strain information of the compliant elements 4011 obtained by the sensitive elements 402 (possibly in the form of electric signals such as resistive or voltage) and ultimately derive a contact force acting on the terminal end of the surgical instrument from the strain information. In this embodiment, the peripheral measurement module 403 may include a microprocessor for receiving the electric signals output from the sensitive elements 402 and computationally deriving the contact force acting on the terminal end of the surgical instrument based on the received electric signals. In place of the microprocessor, any other programmable computing device such as a single chip microcomputer (SCM), PLC controller, an FPGA or the like may be selected. Optionally, the peripheral measurement module 403 is fixedly connected to the power module 301.

FIG. 4b is a structural block diagram of a peripheral measurement module in the surgical instrument according to an embodiment of the present invention. As shown in FIG. 4b, the peripheral measurement module 403 may include, connected to one another in series, a data acquisition unit 4031, a signal conditioning unit 4032 and a calculation and output unit 4033. Specifically, the peripheral measurement module 403 may operate according to the process: the data acquisition unit 4031 first obtains electric signals generated by the sensitive elements 402; the signal conditioning unit 4032 may then condition the electric signals obtained by the data acquisition unit 4031 through subjecting them to a series of processes including, but not limited to, amplification, digital filtering, detrending, removal of outliers, etc, the calculation and output unit 4033 may at last derive the contact force acting on the terminal end of the surgical instrument 3 by analyzing and calculating the conditioned electric signals from the signal conditioning unit 4032 and output the contact force that may be either one-dimensional or multi-dimensional.

FIG. 5a is a schematic diagram of the sleeve 401 disposed over the shaft 302 in the surgical instrument according to an embodiment of the present invention, and FIG. 5b is a schematic enlarged partial view of the sleeve 401 and the shaft 302 of FIG. 5a. As shown in FIGS. 5a and 5b, with the aid of the sleeve 401, the force sensor 400 sleeves over the terminal end of the shaft 302 in the surgical instrument 3. The closer the sleeve 401 is positioned to the end effector 303, the easier the force acting on the terminal end of the surgical instrument 3 can be known. Preferably, the sleeve 401 is flush with an end face of the terminal end of the shaft 302 and connected to the terminal end fixedly by means of an interference fit, insertion, bonding or the like. Alternatively, in order for easier assembly or disassembly to be achieved, it may be connected to the terminal end detachably by means of thread connection, a latch connection, a snap fitting or the like.

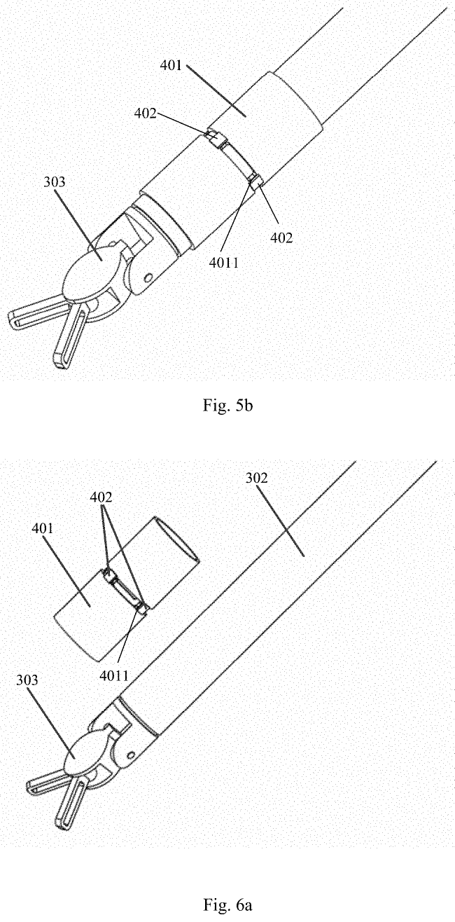

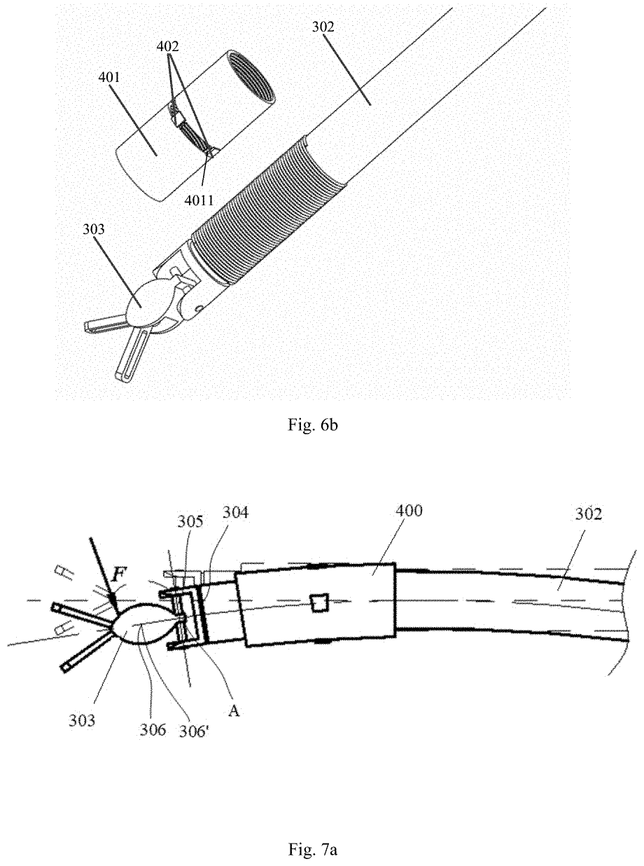

FIG. 6a is a schematic diagram of the sleeve 401 that has not been assembled with the shaft 302 according to an embodiment of the present invention. As shown in FIG. 6a, in this embodiment, the sleeve 401 may be optionally disposed fixedly over the shaft 302 by an interference fit. In this case, the sensitive elements 402 are not limited to being disposed on the outer surfaces of the compliant elements 4011, as they can also be disposed on the inner surfaces of the compliant elements 4011 (e.g., in recesses formed in the inner surfaces of the compliant elements 4011). Obviously, in this embodiment, the interference fit that fixedly connects the sleeve 401 to the shaft 302 can be achieved by the elasticity of the sleeve 401 itself.

FIG. 6b is a schematic diagram of the sleeve 401 that has not yet been assembled with the shaft 302 according to another embodiment of the present invention. As shown in FIG. 6b, in this embodiment, the sleeve 401 may be optionally disposed detachably over the shaft 302 by a threaded fit, with the sensitive elements 402 preferably arranged on the outer surfaces of the compliant elements 4011. In particular, an internal thread may be provided on an inner surface of the sleeve 401, which can make a threaded fit with an external thread formed in an outer surface of the shaft 302. Such a threaded connecting is advantageous in ensuring the absence of any clearance between the sleeve 401 and the shaft 302, which may exert an adverse impact on force measurement accuracy. In case of threaded connecting, in order to ensure reliability of the connecting, the sleeve 401 is preferably made of a material with high hardness, such as carbon fibers.

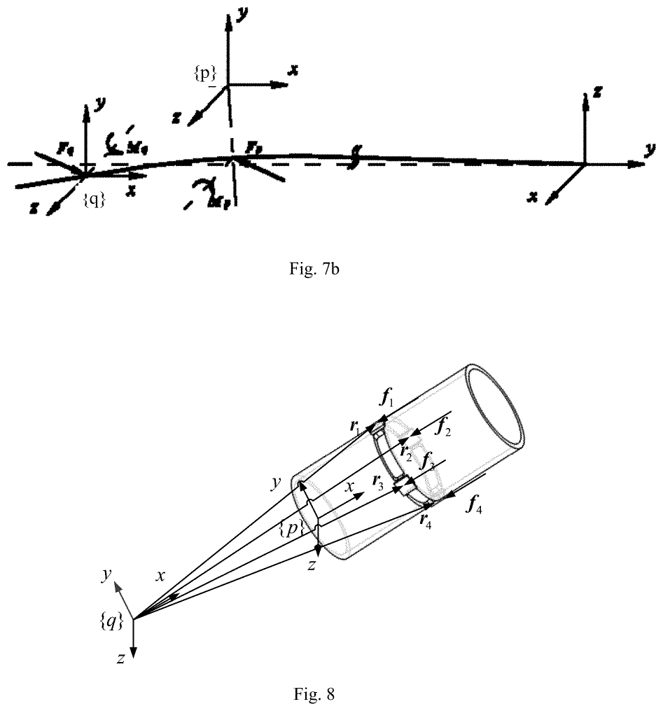

FIG. 7a is a schematic diagram of the surgical instrument that is being stressed on one side and deformed according to an embodiment of the present invention. In this embodiment, the surgical instrument 3 successively includes a roll joint 304, a pitch joint 305 and a yaw joint 306, which allow roll (rotation on its own axis), pitch and yaw motions of the end effector 303, respectively. In this embodiment, the end effector 303 includes two flaps (therefore, the end effector is, for example, scissors). Accordingly, two yaw joints (306, 306′) are required to control a corresponding one of the flaps respectively. As shown in FIG. 7a, upon the end effector 303 being brought into contact with a body tissue, the body tissue will exert a contact force F on the end effector 303. Apparently, this contact force F will be transmitted by the shaft 302 to one end of the sleeve 401 and further to the other end thereof via the compliant elements 4011. Under the action of the contact force F, the compliant elements 4011 will deform as shown in FIG. 7a, and the deformation will be partially sensed by the sensitive elements 402.

FIG. 7b is a schematic diagram illustrating analysis on the force acting on the terminal end of the surgical instrument and measurement principle thereof in accordance with an embodiment of the present invention. As shown in FIG. 7b, when the terminal end of the surgical instrument 3 is brought into contact with a body tissue, the body issue will exert a contact force F on the surgical instrument 3, which may be equivalent to a spatial resultant force F_{q} and a spatial resultant moment M_{q}, which cause the shaft 302 to deform, together with the sleeve 401, as shown in FIGS. 7a and 7b. At the same time, the surgical instrument 3 is subject to a reaction force (Fₚ and Mₚ) from the sleeve 401. Here, the equivalent spatial resultant force F_{q} is balanced with Fₚ, and the equivalent resultant moment M_{q} is balanced with Mₚ. The reaction force that the sleeve 401 applies to the surgical instrument 3 can be obtained by measuring the action force exerted by the surgical instrument 3 to the sleeve 401, and the contact force F exerted by the body tissue on the surgical instrument 3 can be derived from force and moment equilibrium equations.

It will be understood that, in FIG. 7b, which schematically illustrates how the terminal end of the surgical instrument is stressed and deformed, a coordinate system {q} may be defined for the surgical instrument at the terminal end thereof, with an origin located, for example, at the point A marked in the figure, i.e., the intersection of an axis of the shaft 302 and an axis of rotation of the pitch joint 305 in the surgical instrument. The coordinate system {q} also has an x-axis that is perpendicular to the axis of rotation of the pitch joint 305 and coincides with the axis of the shaft 302 in an initial configuration, a y-axis extending parallel to the axis of rotation of the pitch joint 305 in the surgical instrument, and a z-axis that is perpendicular to both the above axes and determined by the right-hand rule. Here, the “initial configuration” is defined as a configuration in which the end effector 303 of the surgical instrument 3 extends straight along the direction of the axis of the shaft 302, with its distal end located farthest away from the yaw joint. The origin of the coordinate system {q} may be the point of application of the equivalent resultant force F_{q} and equivalent resultant moment M_{q}. Since this embodiment is not limited to any particular orientation or position of the sensitive elements 402 on the compliant elements 4011, a coordinate system {p} is desired to be defined for the sleeve 401 as a reference serving to facilitate determining the positions and orientations of the sensitive elements 402, with an origin located for example, at the center of an end face thereof, an x-axis extending along an axis of the sleeve 401, and y- and z-axes being perpendicular to each other and both extending radially. Similarly, the origin of the coordinate system {p} may be the point of application of the resultant force (as well as the associated moment) acting on the sleeve. In addition, ^{q}R denotes the transformation from the coordinate system {p} to {q}, i.e., a descriptor of the posture of the coordinate system {p} in {q}. Since the point of force applied from the surgical instrument 3 to the sleeve 401 is not at the terminal end of the surgical instrument, a position vector rₚ is defined in this embodiment to describe the position of the point of application of the resultant force exerted by the surgical instrument 3 on the sleeve 401 in the coordinate system {q}. That is, it is a descriptor of the position of the coordinate system {p} in {q}. Both the transformation matrix ^{q}R and position vector rₚ are known quantities that have been determined during assembly. For the sake of simple computation, some means are desirably used during assembly to ensure that the coordinate systems {q} and {p} have the same initial orientation, where ^{q}R is an identity matrix, based on which the coordinate systems according to this embodiment are established. Those skilled in the art will appreciate that the positions and orientations of the coordinate systems {q} and {p} can be flexibly selected to meet the actual requirements.

When the sleeve 401 deforms after receiving an action force (−Fₚ and −Mₚ) from the shaft 302, the sensitive elements 402 on the compliant elements 4011 may sense deformations of the compliant elements 4011. The deformations of the compliant elements 4011 and the stress measured by the sensitive elements 402 follow the following relation:

fᵢ=g(εᵢ)=kᵢ·εᵢ+f(εᵢ) i=1,2, . . . ,n, (1-1)

In equation (1-1), i represents the i-th sensitive element out of a total of, for example, n sensitive elements according to this embodiment; fᵢ denotes a stress measured by the i-th sensitive element; εᵢ, the strain of the corresponding compliant element 4011, on which the i-th sensitive element is arranged, along the direction of the sensitive element's sensitive axis; g(⋅), a functional relation between the stress and the strain; kᵢ, a stress-strain factor of the corresponding compliant element 4011 along the direction of the sensitive axis of the i-th sensitive element, which is determined by material parameters of the compliant element 4011; f(εᵢ), a non-linear compensation item for the stress and the strain, which is obtained by calibration. An exemplary calibration method involves: (i) determining whether there is a zero point drift in any of the sensitive elements 402 by checking signals from the sensitive elements 402 when the sleeve 401 is free of any external force; (ii) determining whether the strain of each sensitive element 402 varies non-linearly with an external force load on the sleeve 401 and, if so, including the non-linear compensation term in the above equation; and (iii) ensuring alignment between directions of the calculated force and actually-applied force. Obviously, as used here, the “stress measured by the sensitive element 402” should not be construed as a direct stress reading of the sensitive element 402. Rather, it refers to a stress on a corresponding portion of the sleeve (i.e., the corresponding compliant element 4011) derived according to the above equation (1-1) from a strain of the portion measured by the sensitive element 402.

In addition, information of the force exerted by the shaft 302 on the sleeve 401 contains its direction, which is a representation of the direction of the sensitive axis of the i-th sensitive element in the coordinate system {p}, and the position of its point of application, which is a representation of the position of the i-th sensitive element in the coordinate system {p}. The stress measured by the sensitive element 402, i.e., the action force exerted by the shaft 302 on the compliant element 4011 is represented in the coordinate system {p} as:

(fₓᵢ,f_{yi},f_{zi})^{T}=fᵢeᵢ=fᵢ·(eₓᵢ,e_{yi},e_{zi})^{T} (1-2)

In equation (1-2), eᵢ is a unit vector denoting the representation of the direction of the sensitive axis of the i-th sensitive element (i.e., the direction along which the sensitive element 402 is arranged) in the coordinate system {p}; fᵢ, the stress measured by the i-th sensitive element; fₓᵢ, f_{yi}, f_{zi}, components of fᵢ along the axes of the coordinate system {p}; eₓᵢ, e_{yi}, e_{zi}, components of eᵢ along the axes of the coordinate system {p}; and (⋅)^{T}, a matrix transposition operator.

Further, the direction and position of point of application of the force exerted by the shaft 302 to the sleeve 401 can be represented in the coordinate system {q} as:

fᵢ=^{q}R(fᵢeᵢ) (1-3)

rᵢ=rₚ+^{q}R^{p}rᵢ (1-4)

In equation (1-3), eᵢ denotes a representation of the direction along which the i-th sensitive element 402 is arranged (i.e., the direction of the sensitive axis thereof) in the coordinate system {p}; fᵢ, a representation of the stress measured by the i-th sensitive element 402 in the coordinate system {q}; and ^{q}R, a representation of the posture of the coordinate system {p} in the coordinate system {q}.

In equation (1-4), ^{p}rᵢ denotes a representation of the position of the i-th sensitive element (i.e., the position of the point of application) in the coordinate system {p}; rᵢ, a representation of the position of the i-th sensitive element in the coordinate system {q}; and rₚ a representation of the position of the coordinate system {p} in the coordinate system {q}.

It is to be noted that, in order to ensure a relative position relationship between the sleeve 401 and the shaft 302 of the surgical instrument and hence between the coordinate systems {p} and {q}, grooves or lines can be engraved or drawn on the end face or surface of the sleeve or on the surface of the shaft 302 to facilitate alignment during assembly.

The stresses measured by all the sensitive elements can be combined in the coordinate system {q} to result in:

$$( \begin{matrix}{F}_{q} \\ {M}_{q}\end{matrix} ) = ( \begin{matrix}diag ( 1 \text{/} {n}_{x} , 1 \text{/} {n}_{y} , 1 \text{/} {n}_{z} ) {\sum }_{i = 1}^{n} {f}_{i} \\ diag ( 1 \text{/} {n}_{x}^{′} , 1 \text{/} {n}_{y}^{′} , 1 \text{/} {n}_{z}^{′} ) {\sum }_{i = 1}^{n} {r}_{i} \times {f}_{i}\end{matrix} ) {n}_{x} , {n}_{y} , {n}_{z} , {n}_{x}^{′} , {n}_{y}^{′} , {n}_{z}^{′} \geq 1 \qquad ( 1 \text{-} 5 )$$

As can be seen from equation (1-5), the equivalent resultant force of the contact force acting on the terminal end of the surgical instrument is

$${F}_{q} = diag ( 1 \text{/} {n}_{x} , 1 \text{/} {n}_{y} , 1 \text{/} {n}_{z} ) {\sum }_{i = 1}^{n} {f}_{i} ,$$

and the equivalent resultant moment thereof is

$${M}_{q} = diag ( 1 \text{/} {n}_{x}^{′} , 1 \text{/} {n}_{y}^{′} , 1 \text{/} {n}_{z}^{′} ) {\sum }_{i = 1}^{n} {r}_{i} \times {f}_{i} ,$$

where diag(•) represents a diagonal matrix with elements in the vector • as its diagonal elements, nₓ,n_{y},n_{z} denote the numbers of redundant forces in the x, y, z directions, respectively, and nₓ′,n_{y}′,n_{z}′ are the numbers of redundant moments in the x, y, z directions, respectively.

In order to improve measurement accuracy and reliability, redundancy, i.e., repeated measurement cycles that are deliberately added, may be sometimes introduced into measurement methods used in practical engineering applications. According to the present invention, the number of redundant forces along a certain direction can be interpreted as the number of times the force is measured by a force sensor along the transmission path. Similarly, the number of redundant moments in a certain direction can be interpreted as the number of times the moment is measured by a force sensor along the transmission path. A specific example will be set forth below to describe in greater detail the design of the sleeve-supported force sensor and how it is used to measure a force.

FIG. 8 is a schematic diagram illustrating force measurement principles of a three-dimensional force sensor according to an embodiment of the present invention. FIG. 8 shows the coordinate systems {q} and {p} respectively for the surgical instrument and the sleeve. As noted above, the origin of the coordinate system {q} is arranged at the intersection of the axis of rotation of the roll joint in the surgical instrument 3 (i.e., the axis of the shaft 302) and the axis of rotation of the pitch joint. In addition, the x-axis of the coordinate system {q} is defined as being perpendicular to the axis of rotation of the pitch joint 305 and coincident with the axis of the shaft 302 in the initial configuration, the y-axis as being parallel to axis of rotation of the pitch joint, and the z-axis as being determined by the right-hand rule. Here, the “initial configuration” refers to a configuration in which the end effector of the surgical instrument 3 extends straight along the direction of the axis of the shaft 302, with its distal end located farthest away from the yaw joint. Further, the coordinate system {p} is established on the end face of the sleeve 401. rₚ represents a vector described the translation, and ^{q}R denotes a matrix describing the rotation, from the coordinate system {p} to the coordinate system {q}. Both of them are known quantities determined by an initial mounting position of the sleeve-supported force sensor. For the sake of computational simplicity, it is desirable to ensure, during assembly, that the coordinate systems {p} and {q} are oriented in the same direction so that ^{q}R is an identity matrix. The following analysis is just based on such an orientation of the coordinate systems. In addition, any rotation angle of the surgical instrument at a front end thereof can be measured by an encoder in the power module 301, making ^{q}R still a known quantity.

As shown in FIG. 8, the hollowed-out feature included in the sleeve 401 includes four compliant elements 4011 that are uniformly arranged in the circumferential direction. The force sensor 400 includes four sensitive elements 402, respectively given the subscripts 1 to 4 in the following description for the purpose of distinguishing one from another. In the illustrated example, each one of the four sensitive elements 402 are fixedly arranged on the outer surfaces of a respective one of the compliant elements 4011, and the sensitive axes of the four sensitive elements 402 all extend parallel to the axial direction of the sleeve 401.

The four sensitive elements are arranged, with their sensitive axes being oriented in individual directions indicated as e₁, e₂, e₃, e₄ in the coordinate system {p}. Since all the sensitive elements 402 are oriented in the axial direction of the sleeve 401, each of their directions corresponds to the vector (1,0,0)^{T}. Therefore, according to equations (1-2) and (1-3), the stresses sensed by the four sensitive elements 402 can be represented in the coordinate system {q} as:

$$\{ \begin{matrix}{f}_{1} = {( {f}_{x 1} 0 \; 0 )}^{T} = {f}_{1} {e}_{1} \\ {f}_{2} = {( {f}_{x 2} 0 \; 0 )}^{T} = {f}_{2} {e}_{2} \\ {f}_{3} = {( {f}_{x 3} 0 \; 0 )}^{T} = {f}_{3} {e}_{3} \\ {f}_{4} = {( {f}_{x 4} 0 \; 0 )}^{T} = {f}_{4} {e}_{4}\end{matrix} \qquad ( 1 \text{-} 6 )$$

where fₓ₁, fₓ₂, fₓ₃, fₓ₄ are x-axis components of the stress measured by the first to fourth sensitive elements. It is to be noted that, as the directions of the sensitive axes of these sensitive elements are all parallel to the x-axis, components of the stress measured in the other directions, i.e., the y- and z-axis components, are all zero.

Likewise, the four sensitive elements 402 have respective position vectors in the coordinate systems {p}, indicated as ^{p}r₁, ^{p}r₂, ^{p}r₃, ^{p}r₄, which are determined by the positions where the sensitive elements 402 are arranged. The representations of the sensitive elements 402 in the coordinate system {q}, i.e., r₁, r₂, r₃, r₄ can be obtained from equation (1-4), which are also representations of points of application of the stresses, although the involved equations are not specified herein. The position vectors r₁, r₂, r₃, r₄ are given by:

$$\{ \begin{matrix}{r}_{1} = {( {x}_{1} {y}_{1} {z}_{1} )}^{T} = {r}_{p} + {}_{}^{p}{r}{}_{1}^{} \\ {r}_{2} = {( {x}_{2} {y}_{2} {z}_{2} )}^{T} = {r}_{p} + {}_{}^{p}{r}{}_{2}^{} \\ {r}_{3} = {( {x}_{3} {y}_{3} {z}_{3} )}^{T} = {r}_{p} + {}_{}^{p}{r}{}_{3}^{} \\ {r}_{4} = {( {x}_{4} {y}_{4} {z}_{4} )}^{T} = {r}_{p} + {}_{}^{p}{r}{}_{4}^{}\end{matrix} \qquad ( 1 \text{-} 7 )$$

where x₁, x₂, x₃, x₄, are x-axis coordinates of centers of the first to fourth sensitive elements in the coordinate system {q}, y₁, y₂, y₃, y₄ are y-axis coordinates of the centers of the first to fourth sensitive elements in the coordinate system {q}, and z₁, z₂, z₃, z₄ are z-axis coordinates of the centers of the first to fourth sensitive elements in the coordinate system {q}.

According to the definition of the number of redundant forces along a certain direction made in accordance with equation (1-5), in this embodiment, since each of the x-axial stresses is measured only once during its transmission, nₓ=1. Likewise, according to the definition of the number of redundant moments along a certain direction, in this embodiment, the following conditions are also satisfied: ny′=1, n_{z}′=1. Thus, the contact force acting on the terminal end of the surgical instrument can be obtained, by combining the equations (1-5), (1-6) and (1-7), as:

$$( \begin{matrix}{F}_{q} \\ {M}_{q}\end{matrix} ) = ( \begin{matrix}{f}_{1} + {f}_{2} + {f}_{3} + {f}_{4} \\ {r}_{1} \times {f}_{1} + {r}_{2} \times {f}_{2} + {r}_{3} \times {f}_{3} + {r}_{4} \times {f}_{4}\end{matrix} ) \qquad ( 1 \text{-} 8 )$$

From this equation (1-8), the force F_{qx} acting on the terminal end of the surgical instrument along the x-axis of the coordinate system {q} (axial force) and the moments M_{qy} and M_{qz} along the y- and z-axes of the coordinate system {q} can be obtained as:

$$\{ \begin{matrix}{F}_{qx} = - {f}_{x 1} - {f}_{x 2} - {f}_{x 3} - {f}_{x 4} \\ {M}_{qy} = - {f}_{x 1} {z}_{1} - {f}_{x 2} {z}_{2} - {f}_{x 3} {z}_{3} - {f}_{x 4} {z}_{4} \\ {M}_{qz} = {f}_{x 1} {y}_{1} + {f}_{x 2} {y}_{2} + {f}_{x 3} {y}_{3} + {f}_{x 4} {y}_{4} \end{matrix} \qquad ( 1 \text{-} 9 )$$

As can be seen from equation (1-9), arrangement of the four sensitive elements 402 on the force sensor 400 enables force measurement along one, two or three directions. Specifically, when z₁, z₂, z₃, z₄ are all zero, the moment M_{qy} along the y-axis of the coordinate system will be zero. Similarly, when y₁, y₂, y₃, y₄ are all zero, the moment M_{qz} along the z-axis of the coordinate system will be zero. As a matter of course, x₁, x₂, x₃, x₄ are all none-zero values.

According to this embodiment one or more dimensional force measurement as required can be achieved by first determining coordinates of the sensitive elements in the coordinate system {q} and then determining the positions of the sensitive elements on the sleeve based on the derived coordinates. When one-dimensional contact force measurement is required, y-axis and z-axis coordinates of the sensitive elements may be set to zero. When more, such as two or three, dimensional force measurement is required, y-axis or z-axis coordinates of the sensitive elements, or both, may be set to non-zero values. After that, the sleeve structure can be designed as required so that any undesired portion that is not intended to bear a sensitive element is removed by mechanical processing, thereby forming the hollowed-out feature with the compliant elements 4011 for transmitting a force exerted on the cross-section of the sleeve.

While the measurement principles of the force sensor have been described in detail with reference to the foregoing embodiments, it is a matter of course that the present invention includes, but is not limited to, the above configurations listed hereinabove, and any change made thereto is intended to also fall within the protection scope of the present invention. Other embodiments are available for those skilled in the art in light of the above embodiments.

Further, the force sensor of this embodiment is not limited to the measurement of a three-dimensional force (with a force component in one dimension and moment components in the remaining two dimensions), it may be also applicable to the measurement of a two-dimensional force or even a six-dimensional force (with force components in three dimensions and moment components in the remaining three dimensions). In case of multi-dimensional force measurement, the number of the sensitive elements is at least not lower than the number of dimensions of the contact force. In practice, the process of designing the sensor begins with determination of the number of dimensions of the force to be measured. If the force to be measured is n-dimensional, then at least n independent sensitive elements are necessary. After that, a final force measurement equation (e.g., equation (1-10)) with positions and orientations of the sensitive elements as unknowns is established, and how the sensitive elements are arranged is determined based on the direction of the force to be measured. For example, if the force to be measured is in the x-axis direction, the sensitive elements may be reasonably arranged at positions (with determined x, y, z coordinates) and orientations (with determined fₓ,f_{y},f_{z}), which ensure that a non-zero force measurement can be calculated in said direction. At least, the compliant elements may be designed so as to have the same positions and orientations as the independent sensitive elements. Presented above are merely some basic requirements. In practical engineering applications, in order for improved structural strength and reliability to be achieved, a hollowed-out feature as defined above may be formed across a circumference, with a plurality of (>=2) sensitive elements working in combination to measure a force along a certain direction.

In summary, the force sensor of the present invention is disposed at the terminal end of the surgical instrument and configured to measure a contact force acting on the terminal end. Moreover, the force sensor is provided with a sleeve connected with the surgical instrument, and the sleeve offers at least one of the following advantages:

First, no clearance occurs between the sleeve and the mechanical structure of the surgical instrument. This is conducive to improved measurement accuracy.

Second, for a surgical operation requiring the use of several surgical instruments, a specific calibration process can be performed on each of the surgical instruments, avoiding the influence of instrument material inconsistency on the measurement.

Third, such sleeve can be used on different surgical instruments which transmits deformations of these surgical instruments. Compared to sensitive elements directly attaching to the terminal ends of the surgical instruments, the use of the sensitive elements with the sleeve can avoid measurement errors that may arise from measurement inconsistency between the different sensitive elements on the different surgical instruments, helping in achieving a higher degree of measurement precision and accuracy.

Fourth, coupling the sensitive elements to the surgical instruments with the sleeves can avoid scrappage of the sensitive elements together with the surgical instruments. The surgical instruments are consumable, while the sensitive elements are reusable. Obviously, scraping the sensitive elements together with the surgical instruments is not an economic practice and will lead to increased usage cost.

In a preferred embodiment of the present invention, a plurality of sensitive elements are arranged on the compliant elements of the sleeve in a predetermined manner, and each of the sensitive elements has predetermined coordinates in the coordinate system for the surgical instrument. Based on the predetermined coordinates, the positions of the sensitive elements on the compliant elements can be determined, thus allowing one or more dimensional measurement of a contact force acting on the terminal end of the surgical instrument. This entails an easier and simpler contact force measurement approach, in which one or more dimensional contact force measurement is achievable only by adjusting the positions of the sensitive element relative to the coordinate system for the surgical instrument.

In the present invention, a surgical robot system is further provided. The surgical robot system includes a slave. The slave includes a robotic arm and the surgical instrument as described above. The surgical instrument is detachably connected to a terminal end of the robotic arm and can be driven thereby to move about a remote center of motion (RCM). The surgical robot system also includes a master and a control unit. The master includes a force indicator for showing an information about the contact force acting on the end effector. The control unit is communicatively connected to both the master and the slave and is configured to obtain the information about the contact force acting on the end effector from the force sensor of the surgical instrument and transmit it to the force indicator.

The force indicator may be an imaging system 5 configured to display information about the force acting on the surgical instrument 3. Alternatively, the force indicator may be a master manipulator 7 equipped with a motor. The master manipulator 7 is configured to, upon receiving information about the force acting on the surgical instrument 3, exert a responsive action on the surgeon's hand so that the surgeon can directly “feel” the force acting on the surgical instrument 3.

The slave also includes an endoscope and an endoscope arm detachably connected to the endoscope. In order to facilitate the surgeon to observe how the surgical instrument is being stressed in the field of view of the endoscope, it is necessary to transform the above-described contact force measurement on the terminal end of the surgical instrument into a coordinate system for the endoscope. In this way, a condition of the contact force acting on the terminal end of the surgical instrument can be observed in the field of view of the endoscope (as a force ^{e}F and a moment ^{e}M), allowing the surgeon to make any necessary adjustment to the surgical operation being performed based on the contact force.

By transforming the equivalent resultant force F_{q} and equivalent resultant moment M_{q} acting on the surgical instrument 3 to the coordinate system {e} for the endoscope according to equation (1-10), the contact force F acting on the surgical instrument 3 can be described accordingly:

$$( \begin{matrix}{}^{e} F \\ {}^{e} M\end{matrix} ) = ( \begin{matrix}{}^{e} R & 0 \\ - {S}^{T} ( {}^{e} r ) {}^{e} R & {}^{e} R\end{matrix} ) ( \begin{matrix}{F}_{q} \\ {M}_{q}\end{matrix} ) \qquad ( 1 \text{-} 10 )$$

where ^{e}F denotes an equivalent resultant force in the coordinate system {e}; ^{e}M, an equivalent resultant moment in the coordinate system {e}; ^{e}R, the matrix describing the rotation from the coordinate system {q} to the coordinate system {e} (i.e., the posture of the terminal end of the surgical instrument in the endoscope coordinate system);

^{e}r=(rₓ r_{y} r_{z})^{T}, the position vector from the coordinate system {q} to the coordinate system {e} (i.e., the relative position of the terminal end of the surgical instrument to a terminal end of the endoscope); (⋅)^{T}, a matrix transposition operator; S(^{e}r), an anti-symmetric matrix corresponding to the vector ^{e}r. S(^{e}r) is given by:

$$S ( {}^{e} R ) = ( \begin{matrix}0 & - {r}_{z} & {r}_{y} \\ {r}_{z} & 0 & - {r}_{x} \\ - {r}_{y} & {r}_{x} & 0\end{matrix} ) \qquad ( 1 \text{-} 11 )$$

In this embodiment, an origin of the coordinate system {e} is positioned at the center of the endoscope's field of view. Specifically, the origin of the coordinate system {e} may be located at the terminal end of the endoscope 4. The coordinate system {e} also has an x-axis, a y-axis and a z-axis. The x-axis of the coordinate system {e} is defined as being normal to a lens surface of the endoscope 4 connected to the robotic arm 2. Optionally, an x-axis positive direction of the coordinate system {e} may point from a proximal end of the endoscope 4 (where it is connected to the robotic arm 2) toward the terminal end of the endoscope. The y-axis of the coordinate system {e} is perpendicular to the x-axis thereof and, similarly, the direction of the z-axis of the coordinate system {e} may be determined by the right-hand rule. More preferably, in case of the endoscope being a stereoscopic one, the y-axis of the coordinate system {e} is determined by a line connecting centers of two lens surfaces of the endoscope. Here, since the coordinate system {e} can be established by any suitable technique known in the art, those skilled in the art will recognize, in light of the disclosure herein, how to establish the coordinate system {e} at the endoscope's terminal end and the coordinate system {q} at the terminal end of the surgical instrument, and how to describe, in the coordinate system {e}, the contact force F acting on the surgical instrument in the coordinate system {q} through position and posture transformation between the two coordinate systems.

The description presented above is merely that of a few preferred embodiments of the present invention and does not limit the scope thereof in any sense. Any and all changes and modifications made by those of ordinary skill in the art based on the above teachings fall within the scope as defined in the appended claims.

1. A surgical instrument, comprising a mechanical structure, a sleeve and a force sensor;
wherein the mechanical structure comprises a shaft and an end effector connected to a terminal end of the shaft;
the sleeve fixedly or detachably sleeves over the terminal end of the shaft;
the force sensor comprises at least one sensitive element and a peripheral measurement module connected to the sensitive element, the sensitive element is disposed on the sleeve and configured to obtain a strain information of the sleeve;
the peripheral measurement module is configured to obtain a stress measured by the sensitive element based on the strain information obtained by the sensitive element and obtain both an equivalent resultant force and an equivalent resultant moment of an action force exerted by the shaft on the sleeve based on the stress measured by the sensitive element, the position of the sensitive element and the direction of a sensitive axis of the sensitive element, thereby obtaining a contact force acting on the end effector of the surgical instrument.

2. The surgical instrument of claim 1, wherein the sleeve comprises a hollowed-out feature extending along a circumferential direction, the hollowed-out feature comprising a plurality of compliant elements and openings each located between adjacent two of the compliant elements, the compliant elements being configured to connect an upper section of the sleeve above the hollowed-out feature to a lower section of the sleeve below the hollowed-out feature, and
wherein the at least one sensitive element is arranged on the compliant elements and configured to measure a stress on the compliant elements.

3. The surgical instrument of claim 2, wherein the compliant elements have a modulus of elasticity that is not higher than a modulus of elasticity of the shaft.

4. The surgical instrument of claim 2, wherein each of the compliant elements comprises an inner surface, an outer surface and side surfaces, and wherein the at least one sensitive element is arranged on the inner surfaces, outer surfaces or side surfaces of the compliant elements.

5. The surgical instrument of claim 2, wherein the hollowed-out feature is arranged around an axial middle of the sleeve.

6. The surgical instrument of claim 1, wherein the sensitive axis of the sensitive element is arranged to be parallel or perpendicular to an axial direction of the sleeve.

7. (canceled)

8. (canceled)

9. The surgical instrument of claim 1, wherein the sleeve has one end flush with the terminal end of the shaft.

10. The surgical instrument of claim 1, wherein the peripheral measurement module comprises, communicatively connected to one another in series, a data acquisition unit, a signal conditioning unit and a calculation and output unit; wherein the data acquisition unit is configured to acquire signals output from the sensitive element, the signal conditioning unit is configured to condition the signals output from the sensitive element, and the calculation and output unit is configured to perform a calculation based on the conditioned signals to obtain the contact force on the end effector.

11. The surgical instrument of claim 2, wherein the stress measured by the sensitive element is given by:
fᵢ=g(εᵢ)=kᵢ·εᵢ+f(εᵢ) i=1,2, . . . ,n,
where i represents an i-th sensitive element; fᵢ, a stress measured by the i-th sensitive element; εᵢ, a strain of the compliant element, on which the i-th sensitive element is arranged, along the direction of the sensitive axis of the i-th sensitive element; g(⋅), a function describing a relationship between the stress and the strain; kᵢ, a stress-strain factor of the compliant element, on which the i-th sensitive element is arranged, along the direction of the sensitive axis of the i-th sensitive element; and f(εᵢ), a non-linear compensation term for the stress and the strain.

12. The surgical instrument of claim 11, wherein:
the contact force comprises an equivalent resultant force F_{q} and an equivalent resultant moment M_{q};
the peripheral measurement module is configured to establish a coordinate system {p} for the sleeve thereon, a coordinate system {q} for the surgical instrument at the terminal end thereof, and descriptors of the position and posture of the coordinate system {p} in the coordinate system {q};
the peripheral measurement module is further configured to obtain a descriptor of the stress measured by the sensitive element in the coordinate system {q} based on the descriptor of the posture of the coordinate system {p} in the coordinate system {q}, a descriptor of the direction of the sensitive axis of the sensitive element in the coordinate system {p} and the stress measured by the sensitive element, thereby obtaining the equivalent resultant force F_{q};
the peripheral measurement module is further configured to obtain a descriptor of the position of the sensitive element in the coordinate system {q} based on the descriptors of the position and posture of the coordinate system {p} in the coordinate system {q} and a descriptor of the position of the sensitive element in the coordinate system {p}; and
the peripheral measurement module is further configured to obtain a descriptor of a moment of force acting on the sleeve in the coordinate system {q} based on the descriptor of the stress measured by the sensitive element in the coordinate system {q}, thereby obtaining the equivalent resultant moment M_{q}.

13. The surgical instrument of claim 12, wherein
a descriptor of the stress measured by the sensitive element in the coordinate system {p} is given by:
(fₓᵢ,f_{yi},f_{zi})^{T}=fᵢeᵢ=fᵢ·(eₓᵢ,e_{yi},e_{zi})^{T} i=1,2, . . . ,n,
where eᵢ is a unit vector denoting a descriptor of the direction of the sensitive axis of the i-th sensitive element in the coordinate system {p}; fᵢ, the stress measured by the i-th sensitive element; fₓᵢ, f_{yi}, f_{zi}, components of the fᵢ along axes of the coordinate system {p}; and eₓᵢ, e_{yi}, e_{zi}, components of eᵢ along axes of the coordinate system {p}.

14. The surgical instrument of claim 13, wherein
the descriptor of the stress measured by the sensitive element in the coordinate system {q} is given by:
fᵢ=^{q}R(fᵢeᵢ)
where fᵢ represents a descriptor of the stress measured by the i-th sensitive element in the coordinate system {q}, and ^{q}R represents the descriptor of the posture of the coordinate system {p} in the coordinate system {q}, and wherein
the equivalent resultant force F_{q} is given by:
$${F}_{q} = diag ( 1 \text{/} {n}_{x} , 1 \text{/} {n}_{y} , 1 \text{/} {n}_{z} ) {\sum }_{i = 1}^{n} {f}_{i} ,$$
where diag(•) represents a diagonal matrix with elements in the vector • as diagonal elements, and nₓ, n_{y}, n_{z} represent numbers of redundant forces in the directions of the x-axis, y-axis and z-axis, respectively.

15. The surgical instrument of claim 12, wherein
the descriptor of the position of the sensitive element in the coordinate system {q} is given by:
rᵢ=rₚ+^{q}R^{p}rᵢ
where rᵢ is a descriptor of the position of the i-th sensitive element in the coordinate system {q}; ^{p}rᵢ, a descriptor of the position of the i-th sensitive element in the coordinate system {p}; ^{q}R, the descriptor of the posture of the coordinate system {p} in the coordinate system {q}; and rₚ, a descriptor of the position of the coordinate system {p} in the coordinate system {q}.

16. The surgical instrument of claim 15, wherein
the equivalent resultant moment M_{q} is given by:
$${M}_{q} = diag ( 1 \text{/} {n}_{x}^{′} , 1 \text{/} {n}_{y}^{′} , 1 \text{/} {n}_{z}^{′} ) {\sum }_{i = 1}^{n} {r}_{i} \times {f}_{i} ,$$
Where nₓ′, n_{y}′, n_{z}′ represent the numbers of redundant moments in the directions of the x-axis, y-axis and z-axis, respectively.

17. The surgical instrument of claim 13, wherein:
the hollowed-out feature comprises four compliant elements that are arranged along the circumferential direction; the force sensor comprises four sensitive elements, each arranged on an outer surface of a respective one of the compliant elements; the descriptors e₁, e₂, e₃, e₄ of the directions of the sensitive axes of the four sensitive elements in the coordinate system {p} are all (1,0,0)^{T};
the descriptors of the stress measured by the four sensitive elements in the coordinate system fa are given as:
$$\{ \begin{matrix}{f}_{1} = {( {f}_{x 1} 0 \; 0 )}^{T} = {f}_{1} {e}_{1} \\ {f}_{2} = {( {f}_{x 2} 0 \; 0 )}^{T} = {f}_{2} {e}_{2} \\ {f}_{3} = {( {f}_{x 3} 0 \; 0 )}^{T} = {f}_{3} {e}_{3} \\ {f}_{4} = {( {f}_{x 4} 0 \; 0 )}^{T} = {f}_{4} {e}_{4}\end{matrix}$$
where fₓ₁, fₓ₂, fₓ₃, fₓ₄ respectively represent stress measured by the first to fourth sensitive elements in the direction of an x-axis of the coordinate system {q}; and
the equivalent resultant force F_{q} is given by:
F_{q}=(f₁+f₂+f₃±f₄).

18. The surgical instrument of claim 17, wherein:
^{q}R, the descriptor of the posture of the coordinate system {p} in the coordinate system {q}, is an identity matrix; and
the positions of the sensitive elements are measured as respective center positions, which are described in the coordinate system {q} as:
$$\{ \begin{matrix}{r}_{1} = {( {x}_{1} {y}_{1} {z}_{1} )}^{T} = {r}_{p} + {}_{}^{p}{r}{}_{1}^{} \\ {r}_{2} = {( {x}_{2} {y}_{2} {z}_{2} )}^{T} = {r}_{p} + {}_{}^{p}{r}{}_{2}^{} \\ {r}_{3} = {( {x}_{3} {y}_{3} {z}_{3} )}^{T} = {r}_{p} + {}_{}^{p}{r}{}_{3}^{} \\ {r}_{4} = {( {x}_{4} {y}_{4} {z}_{4} )}^{T} = {r}_{p} + {}_{}^{p}{r}{}_{4}^{}\end{matrix}$$
where ^{p}r₁, ^{p}r₂, ^{p}r₃, ^{p}r₄ are descriptors of the center positions of the four sensitive elements in the coordinate system {p}; rₚ, the descriptor of the position of the coordinate system {p} in the coordinate system {q}; x₁, x₂, x₃, x₄, x-axis coordinates of the center positions of the first to fourth sensitive elements in the coordinate system {q}; y₁, y₂, y₃, y₄, y-axis coordinates of the center positions of the first to fourth sensitive elements in the coordinate system {q}; z₁, z₂, z₃, z₄, z-axis coordinates of the center positions of the first to fourth sensitive elements in the coordinate system {q}; and r₁, r₂, r₃, r₄, descriptors of the positions of the first to fourth sensitive elements in the coordinate system {q}.

19. The surgical instrument of claim 18, wherein
the equivalent resultant moment M_{q} is given as:
M_{q}=(r₁×f₁+r₂×f₂+r₃×f₃+r₄×f₄)

20. A surgical robot system, comprising a slave,
the slave comprising:
a robotic arm; and
the surgical instrument as defined in claim 1,
the robotic arm comprising a terminal end detachably connected to the surgical instrument, the robotic arm being configured to drive the surgical instrument to move about a remote center of motion.

21. The surgical robot system of claim 20, further comprising a master and a control unit, the master comprising a force indicator;
wherein the control unit is communicatively connected to both the master and the slave; the control unit is configured to obtain an information about contact force acting on the end effector from the force sensor of the surgical instrument and transmit the information to the force indicator, the force indicator is configured to show the information about the contact force acting on the end effector.

22. The surgical robot system of claim 20, wherein the slave further comprises:
an endoscope; and
an endoscope arm detachably connected to the endoscope;
wherein the peripheral measurement module of the surgical instrument is configured to establish a coordinate system {p} for the sleeve, a coordinate system {q} for the surgical instrument and a coordinate system {e} for the endoscope, and to obtain, from a matrix ^{e}R describing a rotation from the coordinate system {q} to the coordinate system {e} and a position vector from the r=(rₓ r_{y} r_{z})^{T} coordinate system {q} to the coordinate system {e}, a descriptor of the contact force acting on the end effector in the coordinate system {e} as:
$$( \begin{matrix}{}^{e} F \\ {}^{e} M\end{matrix} ) = ( \begin{matrix}{}^{e} R & 0 \\ - {S}^{T} ( {}^{e} r ) {}^{e} R & {}^{e} R\end{matrix} ) ( \begin{matrix}{F}_{q} \\ {M}_{q}\end{matrix} )$$
where ^{e}F denotes a resultant force in the coordinate system {e}; ^{e}M, a resultant moment in the coordinate system {e}; S(^{e}r), an anti-symmetric matrix corresponding to the vector ^{e}r; F_{q}, the equivalent resultant force of the contact force acting on the end effector; M_{q}, the equivalent resultant moment of the contact force acting on the end effector; and
$$S ( {}^{e} R ) = ( \begin{matrix}0 & - {r}_{z} & {r}_{y} \\ {r}_{z} & 0 & - {r}_{x} \\ - {r}_{y} & {r}_{x} & 0\end{matrix} ) .$$
